(19) United States
(12) Plant Patent Application Publication
(10) Pub. No.: US 2003/0070196 P1
(43) Pub. Date: Apr. 10, 2003

(54) Weigela plant named "Courtanin"

(76) Inventor: Luc Decourtye, Beaucouze (FR)

Correspondence Address: BURNS, DOANE, SWECKER & MATHIS, L.L.P., P.O. Box 1404, Alexandria, VA, 22313-1404, US

(21) Appl. No.: 10/299,810

(22) Filed: Nov. 20, 2002

Related U.S. Application Data
(63) Continuation of application No. 09/667,697, filed on Sep. 22, 2000, now abandoned.

(51) Int. Cl. A01H 5/00

(52) U.S. Cl. PLT/226

(57) ABSTRACT

A new and distinct red-flowered Weigela cultivar is provided that derived by mutagenesis from the ‘Eva Rathke’ cultivar (non-patented in the United States). The growth habit is dwarf, well-rounded, and bushy. Small red blooms with white pistils are formed that are lighter and brighter in coloration than those of the ‘Eva Rathke’ cultivar. Attractive green foliage is formed that is narrower than that of the ‘Eva Rathke’ cultivar and changes to intense red in the fall. The new cultivar is well suited for growing as attractive ornamentation in the landscape. The plant vigor is good and the new cultivar propagates well through the use of softwood cuttings and is well suited for growing in U.S.D.A. Hardiness Zones 4 to 8.

### SUMMARY OF THE INVENTION

A new and distinct cultivar of Weigela florida plant is provided that is a mutation derived from the ‘Eva Rathke’ cultivar (non-patented in the United States).

Plants of the ‘Eva Rathke’ cultivar were irradiated during 1988 with gamma rays derived from cobalt, seeds were formed thereon following open pollination, and the seedlings produced upon the planting of such seeds were observed. The irradiation and the study of the resulting seedlings were carried out at the Institut National De La Recherche Agronomiqul located at Angers, France.

It was found that a single plant observed following such irradiation and the planting of seeds possessed the characteristics of the Weigela cultivar of the present invention. The characteristics of the new cultivar can be summarized as follows:

(a) exhibits a dense, dwarf, well-rounded, erect and bushy growth habit,

(b) forms attractive small red blossoms that are lighter and brighter in coloration than those of the ‘Eva Rathke’ cultivar with white pistils,

(c) forms attractive green foliage that is narrower in configuration than that of the ‘Eva Rathke’ cultivar and changes to intense red in the fall, and

(d) is well suited for growing as attractive ornamentation in the landscape.

The new cultivar of the present invention well meets the needs of the horticultural industry and is particularly well suited for growing at an area where ornamentation is desired at more than one season of the year. During production, the new cultivar is well amenable to growing in containers.

The size of the plant is considerably smaller than that of its ‘Eva Ratlike’ parent. Also, as indicated the blossoms are of a brighter red coloration and the leaves are narrower. One of the smallest Weigela available plants is provided.

The new cultivar has been found to readily undergo asexual propagation by the use of softwood cuttings. Roots readily form on such cuttings. Such asexual propagation has been carried out at Angers, France, and elsewhere.

The new cultivar has been named ‘Courtanin’, and is being marketed under the NAIN ROUGE trademark.

### BRIEF DESCRIPTION OF THE PHOTOGRAPH

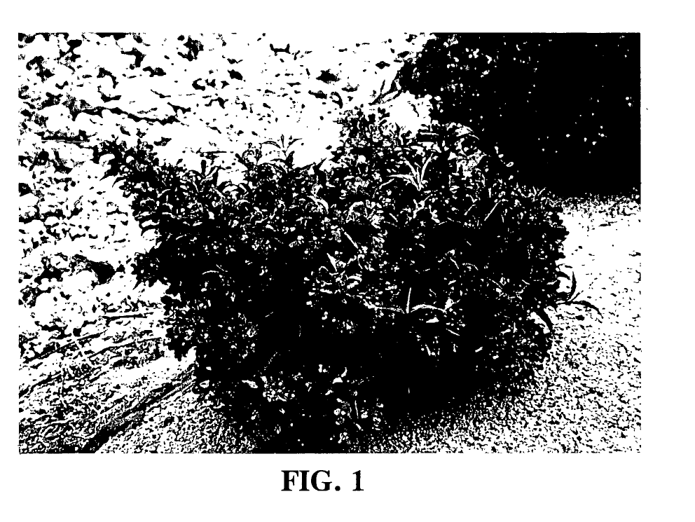

The accompanying FIG. 1 shows a typical plant of the new cultivar when blossoming during the springtime. The plant was grown outdoors at Angers, France. The dense, dwarf well-rounded, erect and bushy growth habit is apparent as well as the profusion of bright red blossoms with light-colored near-white pistils.

### DETAILED DESCRIPTION

The description is based on the observation of mature plants growing outdoors at Angers, France. Such plants had been asexually reproduced through the use of softwood cuttings. Color terminology is provided with reference to the R.H.S. Colour Chart of the Royal Horticultural Society, London. Other common color terms are to be accorded their ordinary dictionary significance.

ORIGIN: Induced mutation utilizing gamma radiation.

PARENTAGE: ‘Eva Rathke’ cultivar (non-patented in the United States).

CLASSIFICATION: Weigela florida, cv ‘Courtanin’.

PLANT: form—flowering shrub.

growth habit.—dense, dwarf, well-rounded, and bushy.

growth rate.—medium to fast.

height.—approximately 1 meter when fully mature at approximately 5 years of age, and considerably less than the ‘Eva Rathke’ cultivar.

width.—approximately 1 meter when fully mature at approximately 5 years of age, and considerably less than the ‘Eva Rathke’ cultivar.

FOLIAGE: disposition—opposite.

color.—medium green changing to intense red in the fall.

configuration.—narrower than the ‘Eva Rathke’ cultivar.

INFLORESCENCE: disposition.—dense in clusters (as illustrated).

configuration.—five-lobed and open widely.

size.—relatively small.

color.—the petals are near Red Group 53 B on the upper surface and near Red Group 53 A on the under surface. This compares to near Greyed-Purple Group 185 B on the upper surface and Red Group 58 A on the under surface for the ‘Eva Rathke’ cultivar. The overall blossom coloration appears to be lighter and brighter than that of the ‘Eva Rathke’ variety.

sepals.—five in number, green in coloration and bear substantial pubescence.

pistils.—light colored, near white, and extend above the petals when the blossoms are fully open.

blooming.—

time.—commonly in May.

HARDINESS: Grows well in U.S.D.A. Hardiness Zone Nos. 4 to 8.

USES: Decorative border, foundation or specimen plant.

1. A new and distinct Weigela cultivar having the following combination of characteristics:
(a) exhibits a dense, dwarf, well-rounded, erect and bushy growth habit,
(b) forms attractive small red blossoms that are lighter and brighter in coloration than those of the ‘Eva Rathke’ cultivar with white pistils,
(c) forms attractive green foliage that is narrower in configuration than that of the ‘Eva Rathke’ cultivar and changes to intense red in the fall, and
(d) is well suited for growing as attractive ornamentation in the landscape. substantially as illustrated and described.
